(19) United States
(12) Patent Application Publication
(10) Pub. No.: US 2015/0352048 A1
(43) Pub. Date: Dec. 10, 2015

(54) VALSARTAN-AMLODIPINE COMPOUND SOLID PREPARATION AND PREPARATION METHOD THEREFOR

(71) Applicants: CHANGZHOU PHARMACEUTICAL FACTORY, Jiangsu (CN); SHANGHAI PHARMACEUTICALS HOLDING CO., LTD., Shanghai (CN)

(72) Inventors: Wenhui LIN, Shanghai (CN); Kepan GAO, Shanghai (CN); Ling FU, Shanghai (CN); Li YING, Shanghai (CN); Chen WU, Jiangsu (CN); Haitao ZHAO, Jiangsu (CN)

(21) Appl. No.: 14/409,719

(22) PCT Filed: Jun. 21, 2013
(86) PCT No.: PCT/CN2013/077633

§ 371 Date: Dec. 19, 2014

(30) Foreign Application Priority Data
Jun. 21, 2012 (CN) 201210209012.6

(51) Int. Cl. A61K 9/14; A61K 9/28; A61K 9/20; A61K 31/4418; A61K 31/41

(52) U.S. Cl. CPC A61K 9/14; A61K 31/4418; A61K 31/41; A61K 9/2054; A61K 9/2027; A61K 9/28

(57) ABSTRACT

The present invention relates to a compound solid preparation and the preparation method therefor and specifically relates to a valsartan-amlodipine compound solid preparation and the preparation method therefor. The valsartan-amlodipine compound solid preparation comprises valsartan particles and an amlodipine premix. The valsartan particles are prepared by preparing wet valsartan particles from a mixture obtained by mixing valsartan and a pharmaceutic adjuvant with an ethanol aqueous solution as a wetting agent and drying the wet valsartan particles. The amlodipine premix is prepared by mixing amlodipine and a pharmaceutic adjuvant. The valsartan-amlodipine compound solid preparation according to the present invention and the preparation method thereof can prevent the two main ingredients, amlodipine and valsartan, from interfering each other, and the preparation method is relatively simple and suitable for large-scale industrial production.

### TECHNICAL FIELD

The present invention relates to a compound solid preparation and the preparation method thereof, and specifically relates to a valsartan-amlodipine compound solid preparation and the preparation method thereof. The valsartan-amlodipine compound solid preparation comprises valsartan particles and an amlodipine premix, wherein, the valsartan particles are prepared by preparing wet particles from a mixture obtained by mixing valsartan and a pharmaceutic adjuvant with an ethanol aqueous solution as a wetting agent, and drying the wet particles; and the amlodipine premix is prepared by mixing amlodipine and a pharmaceutic adjuvant.

### BACKGROUND OF THE DISCLOSURE

With the continuous improvement of human living conditions, hypertension has become one of the most common cardiovascular diseases, and it is also a main cause for the increased morbidity and mortality of congestive heart failure, stroke, coronary heart disease, renal failure, aortic aneurysm and the like. According to a press communique recently published by the International Society of Hypertension, the global population with hypertension or higher blood pressure has reached 972 million, accounting for 26.4 percent of the adult population in the world. In China, the Fourth National Health Services Survey released by the Ministry of Health of China in 2009 showed that circulatory diseases such as heart disease, cerebrovascular disease and hypertension increased significantly, and the number of circulatory disease cases explicitly diagnosed by physicians reached 114 million, of which hypertensive patients increased to 73 million. So far, there is not an effective therapeutic method for radical cure of hypertension in the world, and the blood pressure can only be controlled effectively within a certain range through medications. Consequently, once a person suffers from hypertension, he/she need administer an antihypertensive drug for a lifelong time. Such massive and long-term demand for antihypertensive drug products makes the antihypertensive drugs and preparations thereof continue to weed through the old to bring forth the new.

Valsartan is an orally active specific antagonist of angiotensin (AT) II receptor, which selectively acts on the AT1 receptor subtype. The AT1 receptor subtype reacts to the known effects of angiotensin II, and the AT2 receptor subtype has nothing to do with the cardiovascular effects. Valsartan has no partial agonist activity on AT1 receptor. Amlodipine benzenesulfonate is a dihydropyridine calcium channel antagonist and takes effect by reducing the amount of extracellular calcium ions entered cardiac cells and vascular smooth muscle cells through L-type calcium channel.

From the pharmacological point of view, since the combination of valsartan with amlodipine activates the sympathetic nervous system by the antihypertensive effect of amlodipine and thus increases the dependence of the blood pressure regulation on the renin-angiotensin-aldosterone system, the antihypertensive effect of valsartan is enhanced. In clinical application, a compound preparation with fixed-prescription can reduce administration frequency, alleviate the patients' resistance raised from taking a great amount of drugs, improve the patient's compliance, and achieve control of blood pressure. In addition, it can also reduce side effects caused by the increase of a single dose of drugs.

So far, the clinically applied dosage forms for valsartan and amlodipine or pharmaceutically acceptable salts thereof are mainly tablets and capsules.

CN101237859A discloses a single- or double-layered solid preparation of a combination of valsartan and amlodipine, wherein its preparation method uses a dry granulation method, in which valsartan, amlodipine and pharmaceutically acceptable additives are mixed, compacted by a rolling-compactor, sieved, and milled, followed by compressing the milled materials into tablets.

CN101485657B discloses a valsartan compound preparation and a preparation method thereof, wherein its active drug ingredients are compacted by rolling to obtain a compacted product, which is then sieved to obtain a granular product, and then mixed with a pharmaceutic adjuvant to produce tablets or capsules.

CN101647797B discloses a composition comprising amlodipine benzenesulfonate and valsartan and a preparation method thereof, wherein the preparation method uses a direct compression process of powder.

CN101744813A discloses an amlodipine-valsartan compound solid preparation and a preparation method thereof, wherein amlodipine and valsartan are separately mixed with a suitable pharmaceutic adjuvant, and then the two mixtures are mixed, and granulated by a wet granulation method, dried, and pressed into tablets.

CN101862328B discloses an amlodipine-valsartan capsuled pharmaceutical composition and a preparation method thereof, wherein valsartan and amlodipine are separately mixed with pharmaceutical excipients, added with a suitable amount of a wetting agent to produce a soft material, and sieved to produce wet particles, and then the wet particles are dried, separately sieved to be granulated, and mixed evenly and packed into capsules.

CN101507715B relates to a solid preparation of valsartan and amlodipine and preparation method thereof, wherein valsartan and amlodipine are separately and evenly mixed with suitable pharmaceutical excipients, separately prepared into soft materials with an aqueous solution containing 3% of hydroxypropyl methylcellulose as a wetting agent, granulated, dried and tableted.

### SUMMARY OF THE DISCLOSURE

### Technical Problem

The active pharmaceutical ingredient of valsartan itself has a light density and a poor fluidity, and is easy to produce static electricity, and thus is difficult to be applied in the direct compression process of powder. Moreover, since its dosage is relatively large, it has to be prepared into particles to meet the practical requirements in the production process, so as to ensure a smooth production. Furthermore, The active pharmaceutical ingredient of valsartan is a drug with strong hydrophobicity, and thus has a poor wetting effect during granulation with a conventional binder aqueous solution (e.g. water, starch paste, an aqueous solution of hydroxypropyl methylcellulose, an aqueous solution of polyvidone, an aqueous solution of methylcellulose, an aqueous solution of hydroxypropyl cellulose, an aqueous solution of sodium carboxymethyl cellulose, and an aqueous solution of sodium alginate, etc.) and a large amount of binder is required.

Secondly, since there is a marked difference between the dosages of amlodipine and valsartan in the valsartan-amlodipine compound preparation, and amlodipine is slightly soluble in water, valsartan having a larger dosage could easily affects the dissolution of amlodipine having a smaller dosage, and thus affects the curative effect thereof.

Further, although dry granulation process has a lot of advantages compared with traditional wet granulation process, it has high equipment cost and low equipment penetration.

Therefore, there is a need for a valsartan-amlodipine compound solid preparation and preparation method thereof, in which the mutual interference between the two main ingredients, amlodipine and valsartan, can be avoided and the preparation process is relatively simple and suitable for large-scale industrial production.

### Technical Solution

In one aspect, the present invention provides a valsartan-amlodipine compound solid preparation comprising valsartan particles and an amlodipine premix, wherein the valsartan particles are prepared by preparing wet particles from a mixture obtained by mixing valsartan and a pharmaceutic adjuvant with an ethanol aqueous solution as a wetting agent, and drying the wet particles, and the amlodipine premix is prepared by mixing amlodipine and a pharmaceutic adjuvant.

In the valsartan-amlodipine compound solid preparation of the present invention, the weight ratio of valsartan to amlodipine, based on amlodipine free base, ranges from 10:1 to 50:1, preferably 14:1 to 40:1, and more preferably 80:5 or 160:5.

In the valsartan-amlodipine compound solid preparation of the present invention, the valsartan particles comprise, based on the total weight of the valsartan particles,

(1) valsartan in an amount of 40 to 100% by weight, preferably 60 to 100% by weight, and

(2) a pharmaceutic adjuvant in an amount of 0 to 60% by weight, preferably 0 to 40% by weight, and

the valsartan particles are prepared by preparing wet particles from a mixture obtained by mixing valsartan and a pharmaceutic adjuvant with an ethanol aqueous solution as a wetting agent, and drying the wet particles.

In the valsartan particles, the pharmaceutic adjuvant may be one or more selected from the group consisting of a binder, a filler, a disintegrating agent, a coloring agent and the like. The binder may be one or more selected from the group consisting of polyvidone, hydroxypropyl methylcellulose, hydroxypropyl cellulose, methylcellulose, sodium carboxymethylcellulose, ethylcellulose, polyethylene glycol and the like, and may be used in an amount of 0 to 20% by weight, preferably 0 to 5% by weight, based on the total weight of the valsartan particles. The filler may be one or more selected from the group consisting of starch, microcrystalline cellulose, compressible starch, lactose, mannose, calcium hydrophosphate and the like, and may be used in an amount of 0 to 60% by weight, preferably 0 to 35% by weight, based on the total weight of the valsartan particles. The disintegrating agent may be one or more selected from the group consisting of cross-linked polyvidone, sodium carboxymethylstarch, cross-linked sodium carboxymethylcellulose, low-substituted hydroxypropylcellulose, dry starch and the like, and may be used in an amount of 0 to 10% by weight, preferably 0 to 5% by weight, based on the total weight of the valsartan particles. The coloring agent may be a coloring product in pharmaceutical grade.

In the preparation method of valsartan particles, the ethanol aqueous solution may be one having 1 to 99% by volume, preferably 5 to 30% by volume, most preferably 10% by volume of ethanol. The ethanol aqueous solution may be used in an amount that is enough to sufficiently wet the materials. The wet particles may be prepared by various instruments and methods commonly used in the art, preferably by a high-shear granulator. The drying may be performed by various instruments and methods commonly used in the art, and preferably by a fluidized bed.

In the valsartan-amlodipine compound solid preparation of the present invention, the amlodipine premix comprises, based on the total weight of the amlodipine premix,

(1) amlodipine, based on amlodipine free base, in an amount of 1 to 30% by weight, preferably 2 to 15% by weight, and

(2) a pharmaceutic adjuvant in an amount of 70 to 99% by weight, preferably 85 to 98% by weight, and

the amlodipine premix is prepared by mixing amlodipine and a pharmaceutic adjuvant.

In the present invention, the amlodipine is selected from amlodipine free base and pharmaceutically acceptable salts thereof, such as benzenesulfonate, maleate, hydrochloride, sulfate, acetate and the like, wherein amlodipine benzenesulfonate is preferred.

In the amlodipine premix, the pharmaceutic adjuvant may be one or more selected from the group consisting of filler, disintegrating agent, glidant, coloring agent and the like. The filler may be one or more selected from the group consisting of starch, microcrystalline cellulose, compressible starch, lactose, mannose, calcium hydrophosphate and the like, and may be used in an amount of 40 to 95% by weight, preferably 70 to 90% by weight, based on the total weight of the amlodipine premix. The disintegrating agent may be one or more selected from the group consisting of cross-linked polyvidone, sodium carboxymethyl starch, cross-linked sodium carboxymethylcellulose, low-substituted hydroxypropylcellulose, dry starch and the like, and may be used in an amount of 1 to 30% by weight, preferably 5 to 15% by weight, based on the total weight of the amlodipine premix. The glidant may be one or more selected from the group consisting of aerosil, talc powder and the like, and may be used in an amount of 0 to 5% by weight, preferably 1 to 3% by weight. The coloring agent may be a coloring product in pharmaceutical grade.

In a preferred embodiment, the valsartan particles comprise, based on the total weight of the valsartan particles, (1) valsartan in an amount of 40 to 100% by weight, preferably 60 to 100% by weight, (2) microcrystalline cellulose in an amount of 0 to 60% by weight, preferably 0 to 35% by weight, (3) cross-linked polyvidone in an amount of 0 to 10% by weight, preferably 0 to 5% by weight, and is prepared by preparing wet particles from a mixture obtained by mixing valsartan and a pharmaceutic adjuvant with a 10% ethanol aqueous solution as a wetting agent, and drying the wet particles.

In a preferred embodiment, the amlodipine premix comprises, based on the total weight of the amlodipine premix, (1) amlodipine, based on amlodipine free base, in an amount of 1 to 30% by weight, preferably 2 to 15% by weight, (2) microcrystalline cellulose in an amount of 40 to 95% by weight, preferably 70 to 90% by weight, (3) cross-linked polyvidone in an amount of 1 to 30% by weight, preferably 5 to 15% by weight, (4) aerosil in an amount of 0 to 5% by weight, preferably 1 to 3% by weight, and is prepared by mixing amlodipine and a pharmaceutic adjuvant.

The valsartan-amlodipine compound solid preparation of the present invention may be tablets, capsules or powders, preferably tablets.

In the case of tablets, the valsartan-amlodipine compound solid preparation of the present invention may further contain a disintegrating agent in an amount of 0 to 10% by weight, preferably 0 to 5% by weight; a lubricant in an amount of 0.1 to 5% by weight, preferably 0.5 to 2% by weight, and a coating agent in an amount of 0 to 5% by weight, based on the total weight of the tablets. The disintegrating agent may be one or more selected from the group consisting of cross-linked polyvidone, sodium carboxymethylstarch, cross-linked sodium carboxymethylcellulose, low-substituted hydroxypropylcellulose, dry starch and the like, and preferably sodium carboxymethylstarch. The lubricant may be one or more selected from the group consisting of magnesium stearate, talc powder and the like, and preferably magnesium stearate. The coating agent is preferably Opadry, and the weight gain of coating is preferably 3%.

In another aspect, the present invention provides a method of preparing the valsartan-amlodipine compound solid preparation, comprising:

(1) preparing wet particles from a mixture obtained by mixing valsartan and a pharmaceutic adjuvant with an ethanol aqueous solution as a wetting agent, and drying the wet particles to give valsartan particles;

(2) mixing amlodipine and a pharmaceutic adjuvant to give a amlodipine premix;

(3) mixing the valsartan particles and the amlodipine premix.

Preferably, the method of preparing the valsartan-amlodipine compound solid preparation further comprises a step of separately screening the valsartan and the amlodipine, preferably through a 100-mesh sieve, prior to the preparation.

Preferably, in the step (2) of the method of preparing the valsartan-amlodipine compound solid preparation, the amlodipine and the pharmaceutic adjuvant are mixed and passed through a 60-mesh sieve.

Preferably, the method of preparing the valsartan-amlodipine compound solid preparation further comprises a step of mixing and tableting the valsartan particles, the amlodipine premix, a disintegrating agent and a lubricant, and optionally, further comprises a step of coating.

### Advantageous Effects

1. The invention effectively avoids the adverse effects of valsartan on dissolution of amlodipine by a process separately treating valsartan and amlodipine.

2. The valsartan or the mixture of the valsartan and a suitable pharmaceutic adjuvant is wet-granulated with an ethanol aqueous solution in a certain concentration as a wetting agent, so that the defects that valsartan itself has a light density and a poor fluidity, and is easy to produce static electricity are overcome. In addition, the particle fluidity is improved due to the bridging effect generated by the dissolution of valsartan in ethanol, which decreasing the repose angle from 50.2° before granulation to 29.7°, and thus the requirements of tablet production process are fully met.

3. The valsartan particles obtained by the above wet granulation has a bulk density comparable to that of the amlodipine premix, and thus the amlodipine premix may be fully and uniformly mixed with the valsartan particles directly without granulation, and subsequently tableted and coated. The method not only reduces the used amount of the adjuvant and ensures effective dissolution of the drug, but also can simplify the operation process, improve production efficiency, save energy consumption, and contribute to large-scale industrial production.

### BRIEF DESCRIPTION OF ACCOMPANYING DRAWINGS

FIG. 1 is a graph comparing the dissolution percentage of valsartan and amlodipine in hydrochloric acid between the preparation in Example 3 and an imported preparation, wherein A represents the dissolution curves of amlodipine in the preparations, V represents the dissolution curves of valsartan in the preparations.

FIG. 2 is a graph comparing the dissolution percentage of valsartan and amlodipine in a dissolution medium at pH 6.8 between the preparation in Example 3 and an imported preparation, wherein A represents the dissolution curves of amlodipine in the preparations, V represents the dissolution curves of valsartan in the preparations.

FIG. 3 is a graph showing the plasma concentration-time curves of amlodipine after oral administration of the preparation in Example 3 and an imported preparation in dogs.

FIG. 4 is a graph showing the plasma drug concentration-time curves of valsartan after oral administration of the preparation in Example 3 and imported preparation in dogs.

### DETAILED DESCRIPTION

All the active pharmaceutical ingredients and pharmaceutic adjuvants used in the present invention were commercial available.

The instruments for the experiments included a high-shear granulator, a fluidized bed, a Sotax full-automatic dissolution tester, an ERWEKA bulk density tester, and a Pharm-test powder fluidity tester.

TABLE 1
the summary sheet for the formulations in Examples and Comparative
Examples (g, per 1000 tablets)

| | | | | | | | Com. | Com. |
|---|---|---|---|---|---|---|---|---|
| | Ex. 1 | Ex. 2 | Ex. 3 | Ex. 4 | Ex. 5 | Ex. 6 | Ex. 1 | Ex. 2 |
| Valsartan particles | | | | | | | | |
| Valsartan | 80 | 80 | 80 | 160 | 160 | 160 | 80 | 160 |
| Microcrystalline cellulose | — | 20 | 38 | — | 40 | 76 | | |
| (filler) | | | | | | | | |
| Cross-linked polyvidone | — | 2.5 | 5 | — | 5 | 10 | | |
| (disintegrating agent) | | | | | | | | |
| Wetting agent | 10 Vol. % ethanol solution in suitable amount | | | | | | | |
| Amlodipine premix | | | | | | | | |
| Amlodipine | 6.94 g (5 g, based on the amlodipine free base) | | | | | | | |
| benzenesulphonate | | | | | | | | |
| Microcrystalline cellulose | 68 | 48 | 30 | 136 | 96 | 60 | 68 | 136 |
| (filler) | | | | | | | | |
| Cross-linked polyvidone | 8.5 | 6 | 3.5 | 17 | 12 | 7 | 8.5 | 17 |
| (disintegrating agent) | | | | | | | | |
| aerosil (glidant) | 1.2 | 1.2 | 1.2 | 2.4 | 2.4 | 2.4 | 1.2 | 2.4 |
| Adjuvants for tableting | | | | | | | | |
| Sodium carboxymethylstarch | 3.3 | 3.3 | 3.3 | 6.6 | 6.6 | 6.6 | 3.3 | 6.6 |
| (disintegrating agent) | | | | | | | | |
| Magnesium stearate | 2.5 | 2.5 | 2.5 | 5 | 5 | 5 | 2.5 | 5 |
| (lubricant) | | | | | | | | |

| | |
|---|---|
| Opadry (coating agent) | Coated to 3% weight gain |

Ex.: Example
Com. Ex.: Comparative Examples

### Examples 1-6

According to the formulation in Table 1, valsartan-amlodipine compound tablets of Examples 1 to 6 were prepared respectively by the following preparation method:

(1) valsartan and amlodipine benzenesulphonate were separately screened through a 100-mesh sieve to be ready for use;

(2) the screened valsartan in the step (1) and pharmaceutic adjuvants (microcrystalline cellulose and cross-linked polyvidone) were granulated by a high-shear granulator, and fully wet by adding an appropriate amount of a wetting agent to prepare wet particles, which were dried on a fluidized bed to give valsartan particles to be ready for use;

(3) the screened amlodipine benzenesulphonate in the step (1) and pharmaceutic adjuvants (microcrystalline cellulose, aerosil and cross-linked polyvidone) were mixed and passed through a 60-mesh sieve to give an amlodipine benzenesulphonate premix to be ready for use;

(4) the valsartan particles obtained in the step (2), the amlodipine benzenesulphonate premix obtained in the step (3), sodium carboxymethylstarch as a disintegrating agent and magnesium stearate as a lubricant were mixed to give a mixture;

(5) the mixture obtained in the step (4) was pressed into tablets and coated.

### Comparative Examples 1-2

According to the formulation in Table 1, valsartan-amlodipine compound tablets of Comparative Examples 1 to 2 were prepared respectively by the following preparation method:

(1) Valsartan and amlodipine benzenesulphonate were separately screened through a 100-mesh sieve to be ready for use;

(2) valsartan, amlodipine benzenesulphonate and pharmaceutic adjuvants (aerosil, microcrystalline cellulose and cross-linked polyvidone) were evenly mixed by a high-shear granulator, and added with a wetting agent to be granulated by wet granulation, and then dried on a fluidized bed to give particles;

(3) The particles obtained in the step (2) were mixed with a disintegrating agent and a lubricant to give a total mixture;

(4) The total mixture obtained in the step (3) was pressed into tablets and coated.

### Experimental Example 1

The repose angle was measured on a Pharm-test powder property analyzer (fixed conical bottom method).

The bulk density was measured on a ERWEKA bulk density tester.

Dissolution test is an important indicator for in vitro evaluation on the quality of a preparation. The dissolution test was performed according to the second method in Appendix XC of the second Section of “Chinese Pharmacopoeia 2010”, wherein the dissolved sample was measured by high performance liquid chromatography (HPLC), using 900 ml of a buffer at pH 6.8 as the dissolution medium.

Various properties of the valsartan particles prepared in Examples 1 to 6 and the valsartan-amlodipine compound tablets prepared in Examples 1 to 6 and Comparative Examples 1 to 2 were tested, and the results were shown in Table 2.

TABLE 2
The results of various important properties of the valsartan particles and
the valsartan-amlodipine compound tablets

| | | | | | | | Com. | Com. |
|---|---|---|---|---|---|---|---|---|
| | Ex. 1 | Ex. 2 | Ex. 3 | Ex. 4 | Ex. 5 | Ex. 6 | Ex. 1 | Ex. 2 |
| Repose angle of valsartan | 52.2° | 48.4° | 47.8° | 53.4° | 48.8° | 49.1° | | |
| mixture(before granulation) | | | | | | | | |
| Repose angle of valsartan | 35.1° | 29.7° | 33.7° | 36.0° | 32.4° | 31.1° | | |
| particles | | | | | | | | |
| Bulk density of valsartan | 0.398 | 0.387 | 0.384 | 0.374 | 0.382 | 0.358 | | |
| particles | | | | | | | | |
| Bulk density of amlodipine | 0.338 | 0.346 | 0.349 | 0.343 | 0.359 | 0.339 | | |
| benzenesulphonate premix | | | | | | | | |
| Dissolution percentage for | 92.2% | 93.6% | 93.7% | 93.5% | 90.2% | 91.0% | 76.6% | 78.8% |
| amlodipine at 30 min | | | | | | | | |
| Dissolution percentage for | 97.8% | 101.0% | 99.11% | 98.9% | 95.9% | 98.4% | 95.7% | 94.8% |
| valsartan at 30 min | | | | | | | | |

Ex.: Example
Com. Ex.: Comparative Examples

It is can be seen from Table 2 that, the repose angle of valsartan significantly decreased after granulation, indicating that the granulation of the present invention significantly improved the fluidity of valsartan particles, and the bulk density of the valsartan particles was comparable to that of the amlodipine benzenesulphonate premix, ensuring uniform mixing of the two main ingredients. Furthermore, compared with the 76.6% and 82.8% dissolution percentage for amlodipine at 30 min in Comparative Examples 1 and 2, the dissolution percentage for amlodipine at 30 min in Examples 1 to 6 were more than 90.0% due to the use of a preparation process in which the two active ingredients were treated separately, which effectively avoided the adverse effects of valsartan on dissolution of amlodipine.

### Experimental Example 2

In order to further demonstrate the effect of the valsartan-amlodipine compound preparation of the present invention, comparative tests with respect to the dissolution and in vivo experiment in dogs were carried out with the preparation prepared in Example 3 and an imported preparation. The imported preparation was the commercial valsartan and amlodipine tablets (I) with a trade name of Exforge® (Novartis Pharma Stein AG, Switzerland) widely used in clinic, wherein the dose preparation is 80 mg of valsartan and 5 mg of amlodipine.

The method for measuring dissolution was the same as above.

The in vivo experiment in dogs was carried out as follows. Six male beagle dogs were orally administered with the preparation of Example 3 and the imported preparation, respectively, in a two period crossover study. Blood was collected from the subcutaneous vein in inner side of a foreleg at 0.25, 0.5, 0.75, 1, 1.5, 2, 4, 6, 8, 12, 16, 24, 36, 48, 60, 72 hours after administration, and concentrations of valsartan and amlodipine in the blood were determined by liquid chromatography-tandem mass spectrometry, and the plasma concentration-time curves were plotted.

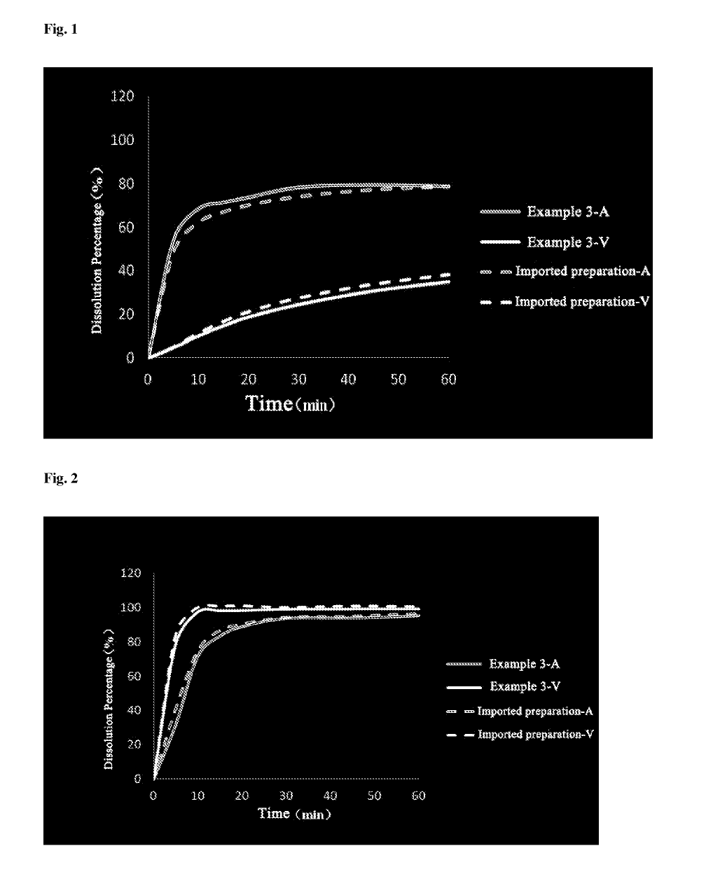

FIG. 1 is a graph comparing the dissolution percentage of valsartan and amlodipine in hydrochloric acid between the preparation in Example 3 and the imported preparation, wherein A represents the dissolution curve of amlodipine in the preparations, and V represents the dissolution curve of valsartan in the preparations. FIG. 2 is a graph comparing the dissolution percentage of valsartan and amlodipine in a medium at pH 6.8 between the preparation in Example 3 and the imported preparation, wherein A represents the dissolution curve of amlodipine in the preparations, and V represents the dissolution curve of valsartan in the preparations. As shown in FIGS. 1 and 2, the dissolution behaviors of amlodipine and valsartan of the preparation in Example 3 are consistent with those of the imported preparation in hydrochloric acid and the dissolution medium at pH6.8. The comparison of the dissolution between the preparation in Example 3 and the imported preparation shows that dissolution curves of the valsartan-amlodipine compound preparation of the present invention are capable of being consistent with the commercial imported preparation.

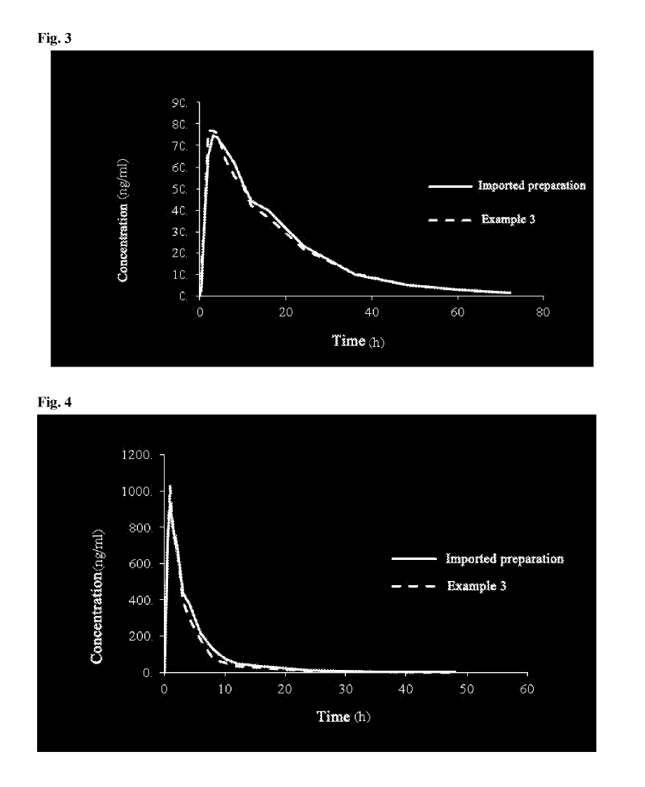

FIG. 3 is a graph showing the plasma concentration-time curves of amlodipine after oral administration of the preparation in Example 3 and the imported preparation in dogs. FIG. 4 is a graph showing plasma concentration-time curves of valsartan after oral administration of the preparation in Example 3 and the imported preparation in dog. As shown in FIGS. 3 and 4, the in vivo plasma concentration-time curves of amlodipine and valsartan in dogs after administration of the preparation in Example 3 are constant with those of the imported preparation. The comparative results of the in vivo experiments in dogs between the preparation in Example 3 and the imported preparation demonstrate that the valsartan-amlodipine compound preparation of the present invention can achieve the same in vivo effects as those of the imported preparation.

Practical Applicability

The present invention wetly granulates valsartan or a mixture of valsartan and a suitable pharmaceutic adjuvant in a high shear granulator by an ethanol aqueous solution in a certain concentration as a wetting agent, and then mixed the resulted valsartan particles with a amlodipine premix. The above process is simple and suitable for large-scale industrial production.

Since valsartan has strong hydrophobicity, the use of ethanol in an appropriate proportion as a wetting agent can effectively improve the wetting properties of the wetting agent on the materials, compared with the water-soluble wetting agent or binder used in a general wet granulation, and thus the amount of wetting agent is reduced and the production efficiency is improved.

1. A valsartan-amlodipine compound solid preparation comprising valsartan particles and an amlodipine premix, wherein,
the valsartan particles are prepared by preparing wet particles from a mixture obtained by mixing valsartan and a pharmaceutic adjuvant with an ethanol aqueous solution as a wetting agent, and drying the wet particles,
the amlodipine premix is prepared by mixing amlodipine and a pharmaceutic adjuvant,
the weight ratio of valsartan to amlodipine, based on amlodipine free base, ranges from 10:1 to 50:1,
wherein the ethanol aqueous solution is one having 5 to 30% by volume of ethanol.

2. The valsartan-amlodipine compound solid preparation according to claim 1, wherein the weight ratio of valsartan to amlodipine, based on amlodipine free base, ranges from 14:1 to 40:1.

3. The valsartan-amlodipine compound solid preparation according to claim 1, wherein the weight ratio of valsartan to amlodipine, based on amlodipine free base, is 80:5 or 160:5.

4. The valsartan-amlodipine compound solid preparation according to claim 1, wherein,
the valsartan particles comprise
(1) valsartan in an amount of 40 to 100% by weight, and
(2) a pharmaceutic adjuvant in an amount of 0 to 60% by weight,
based on the total weight of the valsartan particles;
the amlodipine premix comprises:
(1) amlodipine, based on amlodipine free base, in an amount of 1 to 30% by weight, and
(2) a pharmaceutic adjuvant in an amount of 70 to 99% by weight,
based on the total weight of the amlodipine premix.

5. The valsartan-amlodipine compound solid preparation according to claim 1, wherein,
the valsartan particles comprise
(1) valsartan in an amount of 60 to 100% by weight, and
(2) a pharmaceutic adjuvant in an amount of 0 to 40% by weight,
based on the total weight of the valsartan particles;
the amlodipine premix comprises
(1) amlodipine, based on amlodipine free base, in an amount of 2 to 15% by weight, and
(2) a pharmaceutic adjuvant in an amount of 85 to 98% by weight,
based on the total weight of the amlodipine premix.

6. The valsartan-amlodipine compound solid preparation according to claim 1, wherein,
in the valsartan particles, the pharmaceutic adjuvant may be one or more selected from the group consisting of a binder, a filler, a disintegrating agent, and a coloring agent,
in the amlodipine premix, the pharmaceutic adjuvant may be one or more selected from the group consisting of a filler, a disintegrating agent, a glidant, and a coloring agent.

7. The valsartan-amlodipine compound solid preparation according to claim 1, wherein, the ethanol aqueous solution is one having 10% by volume of ethanol.

8. The valsartan-amlodipine compound solid preparation according to claim 1, wherein, the amlodipine is selected from the group consisting of amlodipine free base and pharmaceutically acceptable salts thereof.

9. The valsartan-amlodipine compound solid preparation according to claim 1, wherein,
the valsartan particles comprise valsartan in an amount of 40 to 100% by weight, microcrystalline cellulose in an amount of 0 to 60% by weight, cross-linked polyvidone in an amount of 0 to 10% by weight, based on the total weight of the valsartan particles;
the amlodipine premix comprises amlodipine, based on amlodipine free base, in an amount of 1 to 30% by weight, microcrystalline cellulose in an amount of 40 to 95% by weight, cross-linked polyvidone in an amount of 1 to 30% by weight, aerosil in an amount of 0 to 5% by weight, based on the total weight of the amlodipine premix.

10. The valsartan-amlodipine compound solid preparation according to claim 1, wherein,
the valsartan particles comprise valsartan in an amount of 60 to 100% by weight, microcrystalline cellulose in an amount of 0 to 35% by weight, cross-linked polyvidone in an amount of 0 to 5% by weight, based on the total weight of the valsartan particles;
the amlodipine premix comprises amlodipine, based on amlodipine free base, in an amount of 2 to 15% by weight, microcrystalline cellulose in an amount of 70 to 90% by weight, cross-linked polyvidone in an amount of 5 to 15% by weight, aerosil in an amount of 1 to 3% by weight, based on the total weight of the amlodipine premix.

11. The valsartan-amlodipine compound solid preparation according to claim 1, wherein, the valsartan-amlodipine compound solid preparation is tablet, capsule or powder.

12. The valsartan-amlodipine compound solid preparation according to claim 11, wherein the valsartan-amlodipine compound solid tablet further comprises a disintegrating agent in an amount of 0 to 10% by weight, a lubricant in an amount of 0.1 to 5% by weight, and a coating agent in an amount of 0 to 5% by weight, based on the total weight of the tablet.

13. The valsartan-amlodipine compound solid preparation according to claim 11, wherein, the valsartan-amlodipine compound solid tablet further comprises a disintegrating agent in an amount of 0 to 5% by weight, a lubricant in an amount of 0.5 to 2% by weight, and a coating agent in an amount of 0 to 5% by weight, based on the total weight of the tablet.

14. A method for the preparing the valsartan-amlodipine compound solid preparation of claim 1, comprising the steps of:
(1) preparing wet particles from a mixture obtained by mixing valsartan and a pharmaceutic adjuvant with an ethanol aqueous solution as a wetting agent, and drying the wet particles to produce valsartan particles,
(2) mixing amlodipine and a pharmaceutic adjuvant to produce an amlodipine premix,
(3) mixing the valsartan particles and the amlodipine premix.

15. The method according to claim 14, further comprising mixing the valsartan particles, the amlodipine premix, a disintegrating agent and lubricant, tableting the mixture, and optionally, coating the tablets.
